(19) United States
(12) Patent Application Publication
(10) Pub. No.: US 2023/0131793 A1
(43) Pub. Date: Apr. 27, 2023

(54) POINT-OF-CARE ASSESSMENT SYSTEM

(71) Applicant: The Curators of the University of Missouri, Columbia, MO (US)

(72) Inventors: Trent M. GUESS, Columbia, MO (US); Rebecca A. BLISS, Columbia, MO (US); Aaron GRAY, Columbia, MO (US); Jamie HALL, Jefferson City, MO (US); Shen_Ying MA, Columbia, MO (US); Brad WILLIS, Columbia, MO (US)

(21) Appl. No.: 17/970,095

(22) Filed: Oct. 20, 2022

Related U.S. Application Data
(60) Provisional application No. 63/270,072, filed on Oct. 21, 2021.

(51) Int. Cl. A61B 5/11; A61B 5/00

(52) U.S. Cl. CPC A61B 5/1128; A61B 5/0077; A61B 5/4082; A61B 5/4088; A61B 5/7475; A61B 2562/0252

(57) ABSTRACT

A system for assessment of neurocognitive and neuromotor control performance, the system comprising a portable force plate configured to collect force plate data indicative of movement and postural control of a subject as the subject performs a task, a depth sensing device configured to, simultaneously with the collection of the force plate data, collect depth data of the subject as the subject performs the task, an interface board configured to, simultaneously with the collection of the force plate data and the collection of the depth data, generate stimuli to instruct the subject to perform a particular task and to generate interface board data indication of input received from the subject in response to the stimuli, and a computer-based controller configured to execute one or more neurocognitive and neuromotor control performance assessment program to analyze the force plate data, the depth data, and the interface board data.

### FIELD

The present teachings relate to system for assessing human motion, and more particularly, for assessing human postural control and movement quality.

### BACKGROUND

The statements in this section merely provide background information related to the present disclosure and may not constitute prior art.

The use of force sensors (e.g., pressures sensors) and force platforms to assess human motion is common. Six-degree of freedom force plates are typically included with instrumented gait analysis software. But these platforms are expensive (e.g., typically greater than $10,000 per platform) and are rigidly attached to the floor. Portable force platforms exist for assessment of athletic performance and balance, but these systems are cost prohibitive for therapy clinics, assisted care facilities, and athletic training centers. Inexpensive 1-dimensional force plates sold through science education distributers can be purchased, but the size and capabilities of these platforms are not optimal for biomechanical assessment.

Motion capture for instrumented gait analysis typically involves specialized labs that use physical markers, visible to motion capture cameras, that are placed on the body. This method is considered the “gold standard” for clinical measurement of human motion, but it is expensive, time consuming, and requires persons skilled in the method. Inertial sensors placed on the body, can also be used to track body motion. While portable, these systems are cost prohibitive for most clinics and still require placement of sensors on the body and expertise for accurate measurement. In various instances, depth cameras (also referred to as depth sensors, or RGBD cameras) have been used to provide an outline of the body that can be used to track body motion and are typically inexpensive, portable, and are marker-less.

### SUMMARY

In various embodiments, the present disclosure provides a point-of-care assessment system (PASS) for the assessment of neurocognitive and neuromotor performance, wherein the system comprises a force plate structured and operable to generate force plate data indicative of measurement of dynamic vertical ground reaction forces on a subject’s body and of the center of pressure of the subject on the force plate as the subject performs one or more tasks. The system additionally comprises an interactive interface board comprising a plurality of subject interface devices, wherein the interface board is structured and operable to provide stimuli to the subject via the subject interface devices, and receive inputs from the subject, via the subject interface devices, in response to the stimuli and generate interface board data indicative of the subject’s inputs simultaneously with at least one of the measurement of the dynamic vertical ground reaction forces and the center of pressure by the force plate and the capturing of the depth image data. The system further comprises a depth image data collection device structured and operable to generate depth image data of the subject as the subject performs the one or more tasks simultaneously with the measurement of the dynamic vertical ground reaction forces and the center of pressure by the force plate and the receipt of subject inputs to the interface board. Still further the system comprises a computer-based data collection and analysis system communicatively connected to the force plate, the depth image data collection device and the interface board, and structured and operable to execute one or more point-of-care assessment system (PASS) program to provide the stimuli and collect and analyze the simultaneously generated force plate data, depth image data, and interface board data.

This summary is provided merely for purposes of summarizing various example embodiments of the present disclosure so as to provide a basic understanding of various aspects of the teachings herein. Various embodiments, aspects, and advantages will become apparent from the following detailed description taken in conjunction with the accompanying drawings which illustrate, by way of example, the principles of the described embodiments. Accordingly, it should be understood that the description and specific examples set forth herein are intended for purposes of illustration only and are not intended to limit the scope of the present teachings.

### DRAWINGS

The drawings described herein are for illustration purposes only and are not intended to limit the scope of the present teachings in any way.

FIG. 1 is an exemplary schematic of a point of care assessment system (PASS), in accordance with various embodiments of the present disclosure.

FIG. 2 is an exemplary illustration of an interactive interface board of the PASS shown in FIG. 1, in accordance with various embodiments of the present disclosure.

FIG. 3A is an exemplary illustration of an image captured by a depth image data collection device of the PASS shown in FIG. 1 as a subject performs a physical activity including body tracking imagery produced by a body tracking algorithm, in accordance with various embodiments of the present disclosure

FIG. 3B exemplarily illustrates the graphical output of Force vs Time and of the center of pressure (COP) generated from force plate data provided by a force plate of the PASS shown in FIG. 1 while the subject is performing the physical activity shown in FIG. 3A, in accordance with various embodiments of the present disclosure.

FIG. 4A exemplarily illustrates data results from a gait test performed using the PASS shown in FIG. 1, in accordance with various embodiments of the present disclosure.

FIG. 4B exemplarily graphically illustrates the PASS assessment of such a gait test performed on older adults, in accordance with various embodiments of the present disclosure.

FIG. 5 exemplarily illustrates the graphical output of the PASS shown in FIG. 1 being utilized to implement various static balance tests, in accordance with various embodiments of the present disclosure.

FIG. 6 is a block diagram of a data collection and analysis system of the PASS shown in FIG. 1, in accordance with various embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements. Additionally, the embodiments disclosed below are not intended to be exhaustive or to limit the invention to the precise forms disclosed in the following detailed description. Rather, the embodiments are chosen and described so that others skilled in the art can utilize their teachings. As well, it should be understood that the drawings are intended to illustrate and plainly disclose presently envisioned embodiments to one of skill in the art, but are not intended to be manufacturing level drawings or renditions of final products and may include simplified conceptual views to facilitate understanding or explanation. As well, the relative size and arrangement of the components may differ from that shown and still operate within the spirit of the invention.

As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims.

Unless otherwise defined, all technical and scientific terms used herein have the same meaning as commonly understood by one of ordinary skill in the art to which this disclosure belongs. The terminology used herein is for the purpose of describing particular example embodiments only and is not intended to be limiting. As used herein, the singular forms “a”, “an”, and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps can be employed.

When an element, object, device, apparatus, component, region or section, etc., is referred to as being “on”, “engaged to or with”, “connected to or with”, or “coupled to or with” another element, object, device, apparatus, component, region or section, etc., it can be directly on, engaged, connected or coupled to or with the other element, object, device, apparatus, component, region or section, etc., or intervening elements, objects, devices, apparatuses, components, regions or sections, etc., can be present. In contrast, when an element, object, device, apparatus, component, region or section, etc., is referred to as being “directly on”, “directly engaged to”, “directly connected to”, or “directly coupled to” another element, object, device, apparatus, component, region or section, etc., there may be no intervening elements, objects, devices, apparatuses, components, regions or sections, etc., present. Other words used to describe the relationship between elements, objects, devices, apparatuses, components, regions or sections, etc., should be interpreted in a like fashion (e.g., “between” versus “directly between”, “adjacent” versus “directly adjacent”, etc.).

As used herein the phrase “operably connected to” will be understood to mean two or more elements, objects, devices, apparatuses, components, etc., that are directly or indirectly connected to each other in an operational and/or cooperative manner such that operation or function of at least one of the elements, objects, devices, apparatuses, components, etc., imparts are causes operation or function of at least one other of the elements, objects, devices, apparatuses, components, etc. Such imparting or causing of operation or function can be unilateral or bilateral.

As used herein, the term “and/or” includes any and all combinations of one or more of the associated listed items. For example, A and/or B includes A alone, or B alone, or both A and B.

Although the terms first, second, third, etc. can be used herein to describe various elements, objects, devices, apparatuses, components, regions or sections, etc., these elements, objects, devices, apparatuses, components, regions or sections, etc., should not be limited by these terms. These terms may be used only to distinguish one element, object, device, apparatus, component, region or section, etc., from another element, object, device, apparatus, component, region or section, etc., and do not necessarily imply a sequence or order unless clearly indicated by the context.

Moreover, it will be understood that various directions such as “upper”, “lower”, “bottom”, “top”, “left”, “right”, “first”, “second” and so forth are made only with respect to explanation in conjunction with the drawings, and that components may be oriented differently, for instance, during transportation and manufacturing as well as operation. Because many varying and different embodiments may be made within the scope of the concept(s) taught herein, and because many modifications may be made in the embodiments described herein, it is to be understood that the details herein are to be interpreted as illustrative and non-limiting.

The apparatuses/systems and methods described herein can be implemented at least in part by one or more computer program products comprising one or more non-transitory, tangible, computer-readable mediums storing computer programs with instructions that may be performed by one or more processors. The computer programs may include processor executable instructions and/or instructions that may be translated or otherwise interpreted by a processor such that the processor may perform the instructions. The computer programs can also include stored data. Non-limiting examples of the non-transitory, tangible, computer readable medium are nonvolatile memory, magnetic storage, and optical storage.

As used herein, the term module can refer to, be part of, or include an application specific integrated circuit (ASIC); an electronic circuit; a combinational logic circuit; a field programmable gate array (FPGA); a processor (shared, dedicated, or group) that performs instructions included in code, including for example, execution of executable code instructions and/or interpretation/translation of uncompiled code; other suitable hardware components that provide the described functionality; or a combination of some or all of the above, such as in a system-on-chip. The term module can include memory (shared, dedicated, or group) that stores code executed by the processor.

The term code, as used herein, can include software, firmware, and/or microcode, and can refer to one or more programs, routines, functions, classes, and/or objects. The term shared, as used herein, means that some or all code from multiple modules can be executed using a single (shared) processor. In addition, some or all code from multiple modules can be stored by a single (shared) memory. The term group, as used above, means that some or all code from a single module can be executed using a group of processors. In addition, some or all code from a single module can be stored using a group of memories.

Referring to FIG. 1, the methodology of measuring reaction time has been well documented for diagnosing post-concussion injury and various other neurocognitive impairments such as dementia. It is most commonly measured using computerized neurocognitive tests but it is unknown whether this measure of reaction time provides sufficient information for accurate analysis of such neurocognitive impairments, e.g., it is unknown whether this measure of reaction time reflects the dynamic reaction time necessary to compete effectively and safely during sport related activities. With regard to concussion analysis, a recent systematic review has shown that there are significant reaction time deficits in the first 3 days following concussion that can linger up to approximately 59 days post injury. In looking at the specific methodology in the large majority of available tests involving concussion diagnosis and analysis what is actually being measured is response time, or total time from stimulus presentation to subject movement completion. Particularly, response time is what has been the focus among both clinicians and researchers as it represents the entire neurocognitive and motor output time needed to complete a specific task. However, such current clinical assessments tend to be simplistic and involve only a single test of manually catching a falling measuring stick, which may not be representative of sport-specific requirements needed during a complex task. Furthermore, normal daily activities require simultaneous cognitive and physical demands and are especially needed in the sport specific realm.

To address this a dual-task assessment paradigm is provided herein to identify performance deficits that can be associated with increased lower extremity injury risk following concussion, and with other neurocognitive impairments such as dementia in the elderly. Higher level neuromotor control deficits that include dual tasking and reaction can identify lingering post-concussion injury and other neurocognitive impairments. Currently there is no one unified platform, system or methodology that simultaneously measures multiple simultaneous inputs received while a subject simultaneously preforms two or more physical and metal tasks. Current management includes collecting symptom inventories, i.e., compiling a list of symptoms collected from one or more separately and independently performed test tasks such as the Balance Error Scoring System (BESS), which is limited by practice, environmental and equipment effects. In addition to the subjectivity of clinical assessment tools, interpreting what constitutes ‘abnormal’ or ‘unusual’ performance can further complicate decision-making when utilizing known neurocognitive assessment systems, methods and platforms.

The present disclosure provides a point-of-care assessment system (PASS) 10 that is structured and operable to address the shortcomings in known concussion and neurological health management. Particularly, the PASS 10 is generally a measurement system that is structured and operable to improve musculoskeletal health through improved methods and technologies for diagnosis and assessment. More particularly, the PASS 10 is structured and operable to detect neuromotor control deficits associated with concussions and other physical and cognitive impairment such as fall risk, stroke, Alzheimer’s, ALS, Parkinson’s disease, etc. Particularly, the PASS 10 utilizes multiple test configurations of functional reaction time, neurocognitive performance and dual (or more) task assessment by examining higher level neuromotor control tasks that are authentic assessments of skills needed for sport activities, performance, normal life skills for the elderly and/or those who may be suffering from dementia or other forms of cognitive impairment or degradation.

The PASS 10 integrates a plurality of measurement technologies with an interactive interface board to assess neuromotor control deficits. The PASS 10 provides a system for assessment of neurocognitive and neuromotor control performance. In various embodiments, the PASS 10 comprises one or more force plate 14, one or more depth image data collection device 18, at least one interactive interface board 22 and a computer-based data collection and analysis system 26 communicatively connected (wired or wirelessly) to the force plate(s) 14, the depth image data collection device(s) 18, and the interface board(s) 22. In various embodiments, the PASS 10 can additionally comprise one or more digital, RGB and/or CCD 2D (2-dimensional) camera 30 that is communicatively connected (wired or wirelessly) to the data collection and analysis system 26 to collect 2D (2-dimensional) image data. The depth image data collection device(s) 18 can be one or more depth camera and/or one or more LiDAR (light detection and ranging) device and/or one or more of any other device structured and operable to collect depth imaging data. Generally, with regard to tracking body motion, the depth image data collection device(s) 18 are coupled with algorithms that identify anatomical features (e.g., elbow joint) in the depth image generated by the depth image data collection device(s) 18. In various embodiments, the force plate(s) 14 can be one or more portable force plate, the interface board(s) 22 can be one or more portable interface board, the depth image data collection device(s) 18 can be one or more portable depth image data collection device, the 2D camera(s) 30 can be one or more portable 2D camera, the computer-based data collection and analysis system 26 can be a portable computer-based data collection and analysis system, and all other components of the PASS 10 can be portable such that the PASS 10 is a portable system.

Particularly, in various embodiments, the PASS 10 provides a portable, inexpensive measurement platform for on-site instrumented assessment of postural control and movement quality that is a useful tool for assessment of balance and human motion for multiple applications including, but not limited to, 1) concussive injuries, 2) post-op rehabilitation, 3) musculoskeletal injury risk, 4) athletic performance, 5) developmental disabilities, 6) fall risk screening, and 7) dementia detection and analysis. In such embodiments, the PASS 10 includes portable technologies that can assess multiple dynamic balance and movement tests including center of pressure, dual limb ground reaction forces, motion of body segments, and reaction time for use in a variety of clinically important assessments. For example, in various instances the PASS 10 can be a lightweight and portable point-of-care devices that can be used to objectively measure concussion-related impairments and improve injury detection and critical decision-making including safe return to sport, work, or activity protocols.

The force plate(s) 14 is/are structured and operable to measure dynamic vertical ground reaction forces on a subject’s body (e.g., gravitational forces on each limb) and center of pressure during single, dual or more task assessments and communicate the collected reaction force and center of pressure data to the data collection and analysis system 26. The depth image data collection device(s) 18 is/are structured and operable to track and measure body motion in each of the X, Y, Z coordinate system axis and communicate the depth image data to the data collection and analysis system 26 simultaneously with the collection of force plate data and/or all other tasks being simultaneously performed by the subject. In various embodiments, the depth image data can be combined with skeletal tracking software (e.g., a skeletal tracking algorithm) to measure body motion in the form of joint centers and segment orientations simultaneously with the collection of force plate data and/or all other tasks being simultaneously performed by the subject. The interface board(s) 22 is/are structured and operable to receive commands from the data collection and analysis system 26 that cause the interface board(s) 22 to provide programmed physical, visual and/or audible stimuli to the subject, simultaneously with the collection of force plate and/or depth image data and/or all other tasks being simultaneously performed by the subject. Additionally, the interface board(s) 22 is/are structured and operable to receive physical, optical and/or audible response input from the subject in response to the programmed physical, visual and/or audible stimuli and communicates this response data to the data collection and analysis system 26 simultaneously with the collection of force plate and/or depth image data and/or all other tasks being simultaneously performed by the subject.

More particularly, the interface board(s) 22, is/are controlled by the data collection and analysis system 26 that executes one or more PASS program and/or algorithm (referred to herein as the PASS programs) that control operation of the interface board(s) 22 to induce physical and cognitive task activities (e.g., single, dual or more task activities) and measure reaction times. Furthermore, the data collection and analysis system 26, via execution of the PASS programs, collects and analyzes the data received from the force plate(s) 14, the depth image data collection device(s) 18, and the 2D image data 30. For example, in various embodiments, the data collection and analysis system 26 can comprise one or more microcontroller that is structured and operable to execute PASS programs to induce cognitive task activities (e.g., single, dual or more task activities) for the subject to preform via interaction with a plurality of subject interface devices 34 disposed on the interface board(s) 22 (e.g., lights, sensors, push buttons, touch screen display buttons, a microphone, switches, etc.,) and measure reaction times, as described further below. In various instances, the PASS programs include various task algorithms designed to measure reaction time and/or implement dual task activities wherein stimuli such as lights and/or audio stimuli requiring a specific responsive action (e.g., a specific audio response or a specific physical action) are provided to a subject simultaneously with the subject preforming an instructed physical activity. The resulting reaction time data and dual task data is collected, stored and analyzed by the data collection and analysis system 26.

In various embodiments, it is envisioned that the interface board 22 can comprise a virtual interface using VR. In such instances the interface board 22 would have virtual buttons that could be pressed for reaction time tests and could be used to provide information to the user in regards to dual tasking. (e.g., a math problem could be displayed during balance test). In such instances, the PASS 10 set up would be augmented reality, where an image of the room is passed through, but augmented with virtual buttons.

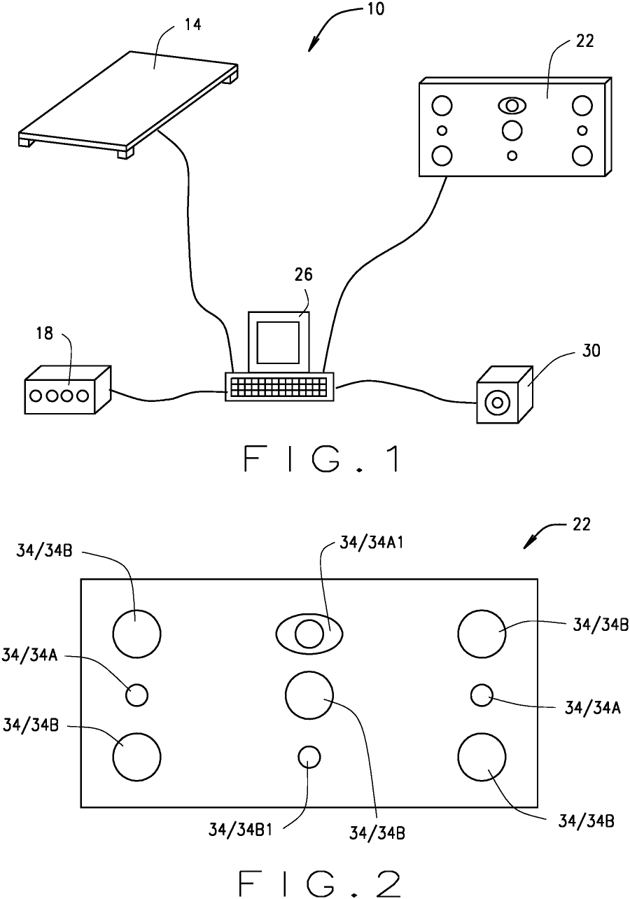

Referring now to FIGS. 1 and 2, the interface board(s) 22 comprises a plurality of subject interface devices 34. In various embodiments, the subject interface devices 34 can comprise one or more subject stimulus device 34A (e.g., lights, e.g., a plurality of different colored lights or LEDs) and one or more user/subject input device or sensor 34B with which the user interacts (e.g., such as a plurality of different colored push buttons and/or different colored illuminating push buttons and/or different colored touch screen display buttons and/or different colored switches, or any other type of mechanical or electrical or electro-mechanical button or switch). Additionally, in various embodiments the subject stimulus devices 34A can include a speaker 34A1 or any other suitable stimulus device, and the input devices 34B can include a microphone 34B1 or any other suitable input device.

An example of a reaction time program can include a subject activating an input device 34B (e.g., pressing a touch screen button or a physical button) that elicits activation of a subject stimulus device 34A (e.g., elicits a particular one of the different colored lights 34A (as selected by the PASS programs) to illuminate) and then as fast as possible activating a specific different input device 34B (e.g., pressing a specific touch screen or physical button) that is dictated by or based on the stimulus provided by the stimulus device 34A (e.g., based on the color of the illuminated light). Another example can be to instruct a subject to press a particular color input device 34B upon activation of a stimulus device 34A (e.g., upon the illumination of a particular color light). For example, instruct the subject to press a red button on the right side of the interface board 22 when a green light is illuminated on the left side of the interface board 22, and press a green button on the left side of the interface board 22 when a red light is illuminated on the right side of the interface board 22. Reaction time from when the stimulus device 34A is activated (e.g., from when the light is illuminated) to when the appropriate input device 34B is activated can be measured, stored and analyzed by the data collection and analysis system 26. Variations of this task can include multiple combinations of activating various input devices 34B that results in activation of a stimulus device 34A (e.g., the illumination of different ones of the colored lights), whereby the subject is instructed to press selected different ones of others of the input devices 34B based on the stimuli provided by the stimulus device 34A (e.g., based on the color of the illuminated light).

An example of a fall risk and/or cognitive impairment evaluation test can be to have subject stand on the force plate 14 and while monitoring their movement via the depth image data collection device 18 and/or the 2D camera 30, have the subject reach down and pick up and object. The data of how the subject controls their body during performance of the task is collected from the force plate 14, depth image data collection device(s) 18 and/or the 2D camera(s) 30, stored and analyzed by the data collection and analysis system 26. Another example can be to have a plurality of buttons on the floor (e.g., one red and one green button), and have the control interface board 22 (via execution of the PASS program by the data collection and analysis system 26) indicate which color button the subject should reach down and push. Then, as the subject executes the task, measure, collect, store and analyze movement data (e.g., dynamic vertical ground reaction force and center-of-pressure (COP)) via the force plate(s) 14, the depth image data collection device(s) 18, and/or the 2D camera(s) 30. In various instances, other data such as the time it takes the subject complete the task can be measured, collected, stored and analyzed.

In various embodiments, the PASS 10 can further utilized a marker base motion capture where a plurality of sensors that are disposed on the subject and sensor outputs are measured by the depth image data collection device(s) 18, whereafter the collected depth image data can be converted into joint angles and joint center measurements to assess spatiotemporal parameters and data. Additionally, it is envisioned that in various embodiments, the PASS can include a heart rate monitor and/or blood pressure monitor to provide additionally biometric data to be collected and analyzed by the PASS programs.

Dual task activity testing can be measuring dynamic vertical ground reaction force and COP via the force plate(s) 14, and collecting movement and coordination data via the depth image data collection device(s) 18, and/or the 2D camera(s) 30 as the subject in involved in memory games such as “Simon Says”, or having the subject perform mental tasks such as adding and subtracting while marching in place, or bending over to touch their toes, or pushing a specific sequence of buttons on the floor or the interface board 22. The interface board 22 can also provide audio commands, such as “press the green button on the right side of the interface board” or “count backwards from 100 by 7”. In such instances the dual task activity test can be automated and standardized. Accordingly, cognitive abilities can be measured simultaneously with quantification of balance control in various static positions and/or dynamic movement activities. In various other embodiments, the PASS program can implement and execute speech recognition tasks, via the speaker 34C and/or microphone 34D, such as audibly instructing (e.g., verbally commanding) a subject to count backwards while pushing a particular sequence of colored buttons 34B. Another dual task activity can be a drop and cut assessment where a subject stands on a box having a pressure sensor that triggers selected ones of the stimulus devices 34A (e.g., selected lights on the interface board to illuminate) when the subject jumps off the box to land on a force plate 14. As the subject jumps off the box, a stimulus device 34A, as selected by the PASS program, is activated (e.g., one of the lights illuminate). Prior to beginning the test the subject has been informed that if a particular stimulus is provided (e.g., a red light illuminates) he/she is to land on the force plate cut (or side step) in a particular direction (e.g., left), and if a different particular stimulus is provided (e.g., a green light illuminates) he/she is to land on the force plate cut (or side step) in a different particular direction (e.g., right). In such a duel task test various data can be collected such as the time to complete the jump and cut, the force during the land and cut (via the force plate 14), the COP during the land and cut (via the force plate 14), the subject’s posture during the jump and cut (via the depth image data collection device 18 and/or the 2D camera), and the subject’s joint angles, joint centers and segment orientations (via the via the skeletal tracker algorithm overlayed on the depth image data).

As set forth herein, the PASS 10 is structured and operable to measure and assess motor control by measuring dynamic vertical ground reaction force and COP via the force plate(s) 14 and collecting movement and coordination data, via the depth image data collection device(s) 18 and/or the 2D camera(s) 30 during any one or more of a large plurality of specific single and dual (or plural) activity tasks as set forth above. Additional single and dual (or plural) activity task tests can comprise the measurement, collection, storage and analysis of dynamic vertical ground reaction force, COP and movement and coordination data during the performance of such tests as the Romberg balance tasks, lateral step down tasks, drop vertical jump tasks, gait tests, step-down tasks, sit-to-stand tasks, upper extremity pointing tasks etc. Selection of task to be assessed can be based on the age, population, physical and/or mental condition, specific injury the subject has incurred, the specific injury at which the subject is thought to be at risk of incurring, and any other category or class of subject. For example, the PASS task and test selection for an elderly subject at risk of falling, would be different than the PASS task and test selection for an athlete recovering from injury or concussion.

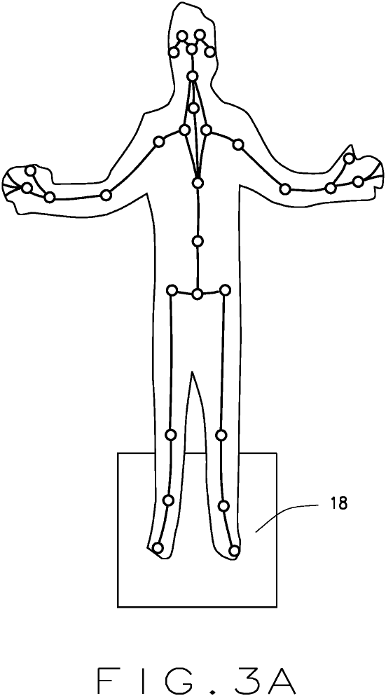

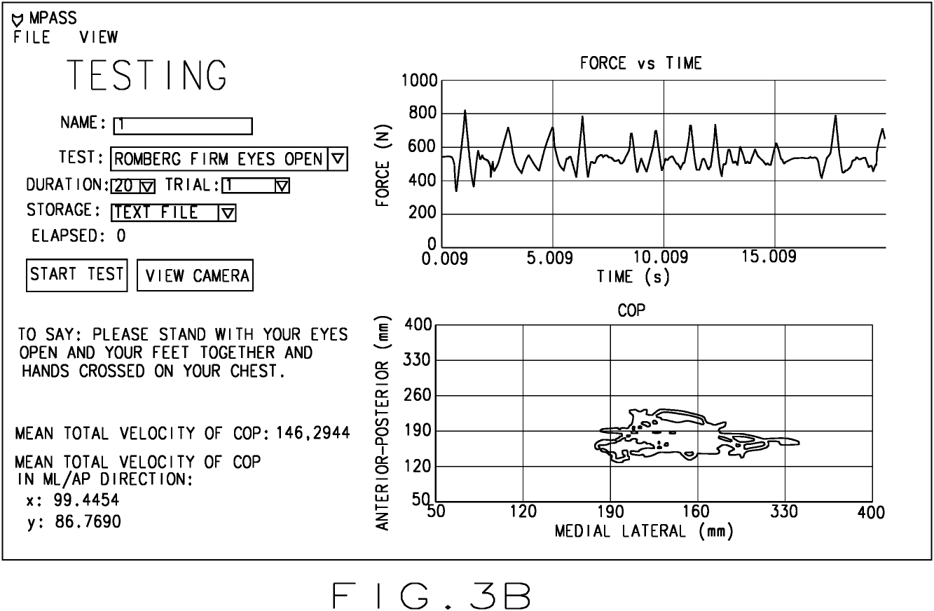

Referring now to FIGS. 3A and 3B, as described above, the PASS 10 is structured and operable to assess movement and postural control of the subject during performance of a selected task (e.g., single and/or dual activity task) or a sequence of tasks (e.g., single and/or dual activity tasks). Particularly, the force plate(s) 14 are structured and operable to measure dynamic vertical ground reaction force and center-of-pressure (COP) of a subject as the subject preforms a selected task or sequence of tasks (e.g., single and/or dual activity tasks), and the depth image data collection device(s) 18 and/or 2D camera(s) 30 are structured and operable to collect movement data of the subject. As exemplarily illustrated in FIGS. 3A and 3B, in various embodiments, the depth camera(s) 18 collect(s) movement data and provides an outline of subject’s body. Additionally, in various instances, the PASS program can include a body tracker algorithm that fits a skeleton to the outline of the body, and thereby can provide joint location and segment orientation information for the subject. Such joint location and segment orientation data can then be converted into joint angles (e.g., hip and knee flexion angles), joint center measurements such as medial knee position, and spatiotemporal parameters of gait. FIG. 3A exemplarily illustrates the output of the body tracker algorithm overlaid on the depth image data from the depth image data collection device 18 of a subject preforming a physical activity (e.g., moving hands in a circle) while standing on the force plate 14. FIG. 3B exemplarily illustrates the graphical output generated from the force plate dat of Force vs Time and of the COP while the subject is performing the physical activity.

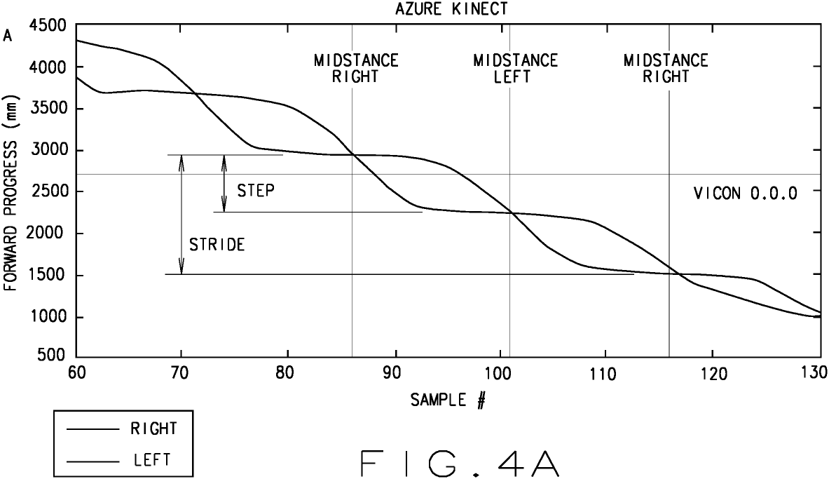

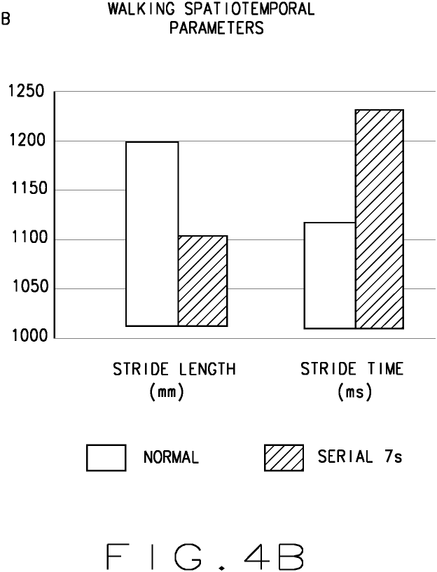

Referring now to FIGS. 4A and 4B the PASS 10 can be implemented to analyze a subjects gait to determine various neuromotor and neurocognitive impairments and/or states of recovery. For example, a subject can be asked to walk across a plurality of force plate 18 while the PASS 10 collect force plate data and/or depth image data (with or without skeletal tracking) and/or 2D image data. FIGS. 4A and 4B show exemplary data results from such a gait test. For example, forward progression of right and left ankle joint centers during walking are graphically illustrated in FIG. 4A, wherein the PASS programs automatically extract walking spatiotemporal parameter from ankle joint center motion. Similarly, FIG. 4B exemplarily graphically illustrates the PASS 10 assessment of older adults wherein decreased stride length is and increased stride time is revealed. Decreased stride length and increased stride time with dual tasking would be an example of output measurements that could be used for evaluation.

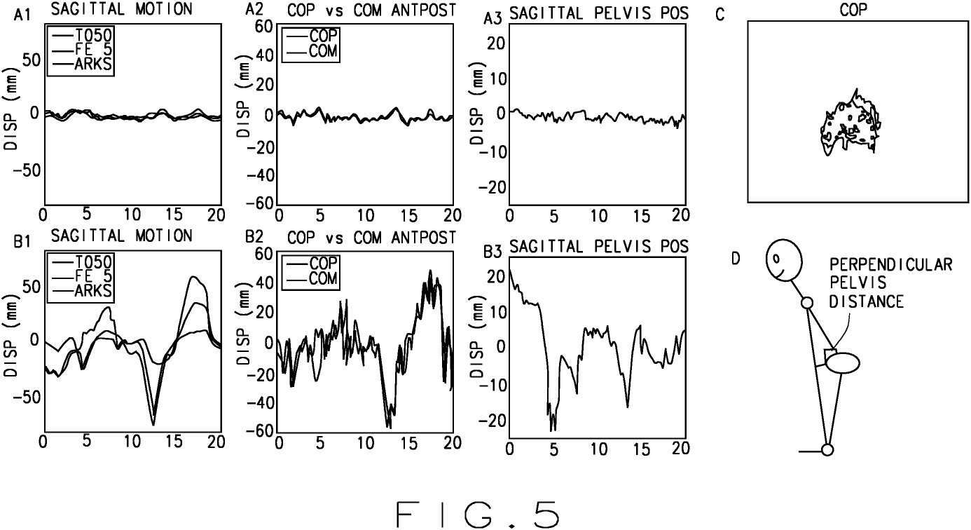

Referring to FIG. 5, which exemplarily illustrates the graphical output of the PASS 10 utilized to implement a static balance test (e.g., Romberg test) on an older adult. The three graphs A1, A2 and A3 exemplarily illustrate the results of the subject standing on a firm surface with eyes open, and the three graphs B1, B2 and B3 exemplarily illustrate the results of the subject standing on foam with eyes closed. Waveforms include sagittal plane motion of body segments, anterior/poster center of pressure (COP) and body center of mass (COM), and perpendicular pelvis distance. The graph C exemplarily illustrates the center of pressure (COP) and the center of mass (COM) waveforms generated from the force plate data, where medial/lateral motion is plotted against anterior/posterior motion. This tracks the motion of COP and is a common graph provided with force plate balance test. The illustration D exemplarily illustrates the perpendicular pelvis distance, which is the distance from the pelvis to a line connecting the torso and ankles, derived from the depth image data collection device 18.

The analysis of the collected data from the single and dual task tests illustrated and/or described herein can be indicative of the acuity and fitness of various physical and/or mental parameters a subject. Moreover, such collected data can be indicative physical and/or mental limits and/or impairments of a subject that can be helpful in deriving a proper, effective and timely recovery plan for the patent. For example, a simple gait task test implemented and executed by the PASS 10 may indicate spatiotemporal deficits in post-concussion individuals such as more conservative gait characteristics exhibited in decreased gait speed, dynamic instability as measured by COP movement in relation to a base of support as well as foot placement indicating increased sway during ambulation. When adding in a dual task test, gait deviations can be increased in post-concussive individuals and the inability to adequately perform both tasks can be compromised. Spatiotemporal deficits in dual task tests can include slower average walking speed, shorter stride length, great double leg stance support time, increased medial-lateral displacement, and faster peak medial lateral velocity. Although only certain system interfaces, e.g., the force plate(s) 14, the interface board(s) 22, the depth image data collection device(s) 18 and the 2D camera(s) 30 are described herein, it is envisioned that any other type of interface can be added to PASS 10 to collect, store and analyze additional data utilized to measure and analyze the acuity and fitness of various physical and/or mental parameters a subject as described above.

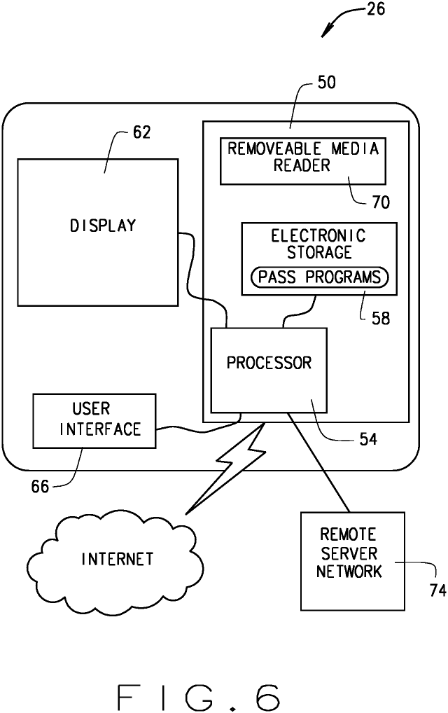

Referring now to FIG. 6, as described above, the PASS 10 is operated and controlled by the data collection and analysis system 26. More particularly, the PASS 10 is operated and controlled by execution of various PASS software, programs, algorithms, and/or code (referred to herein as the PASS programs) executed by at least one processor of the data collection and analysis system 26. In various embodiments, the data collection and analysis system 26 includes various computers, controllers, programmable circuitry, electrical modules, etc. that can be located at various locations of the PASS 10. More specifically, in various embodiments, the data collection and analysis system 26 is a computer-based system that can include one or more computers and/or computer-based modules 50 that each include at least one processor 54 suitable to execute the PASS programs (e.g., the various software, programs, algorithms, and/or code) that control all automated functions and operations of the PASS 10, as described herein. Each computer based-module 50 can additionally include at least one electronic storage device 58 that comprises a computer readable medium (e.g., non transitory, tangible, computer readable medium) such as a hard drive, erasable programmable read-only memory (EPROM), electronically erasable programmable read-only memory (EEPROM), read-write memory (RWM), etc. Other, non-limiting examples of the non-transitory, tangible, computer readable medium are nonvolatile memory, magnetic storage, and optical storage. Generally, the electronic storage device(s) 58 can be any electronic data storage device for storing such things as the various software, programs, algorithms, code, digital information, data, look-up tables, spreadsheets and/or databases, etc., used and executed during operation of the PASS 10, as described herein.

Furthermore, in various implementations, the data collection and analysis system 26 can include at least one display 62 for displaying such things as information, data and/or graphical representations, and at least one user interface device 66, such as a keyboard, mouse, stylus, and/or an interactive touchscreen on the display 66. In various embodiments, some or all of the computer-based modules 50 can include a removable media reader 70 for reading information and data from and/or writing information and data to removable electronic storage media such as floppy disks, compact disks, DVD disks, zip disks, flash drives or any other computer readable removable and portable electronic storage media. In various embodiments the removable media reader 70 can be an I/O port of the respective computer-based module 50 utilized to read external or peripheral memory devices such as flash drives or external hard drives.

In various embodiments, the data collection and analysis system 26 can be communicatively connectable to a remote server network 74, e.g., a local area network (LAN), via a wired or wireless link. Accordingly, the data collection and analysis system 26 can communicate with the remote server network 74 to upload and/or download data, information, algorithms, software programs, and/or receive operational commands. Additionally, in various embodiments, the data collection and analysis system 26 can be structured and operable to access the Internet to upload and/or download data, information, algorithms, software programs, etc., to and from Internet sites and network servers. In various embodiments, the PASS programs executed by the processor(s) 54 to control the operations of the PASS 10 can be top-level system control software that not only controls the discrete hardware functionality of the PASS 10, but also prompts an operator for various inputs.

The description herein is merely exemplary in nature and, thus, variations that do not depart from the gist of that which is described are intended to be within the scope of the teachings. Moreover, although the foregoing descriptions and the associated drawings describe example embodiments in the context of certain example combinations of elements and/or functions, it should be appreciated that different combinations of elements and/or functions can be provided by alternative embodiments without departing from the scope of the disclosure. Such variations and alternative combinations of elements and/or functions are not to be regarded as a departure from the spirit and scope of the teachings.

What is claimed is:

1. A point-of-care assessment system for the assessment of neurocognitive and neuromotor performance, said system comprising:
a force plate structured and operable to generate force plate data indicative of measurement of dynamic vertical ground reaction forces on a subject’s body and of the center of pressure of the subject on the force plate as the subject performs one or more tasks;
an interactive interface board comprising a plurality of subject interface devices, wherein the interface board is structured and operable to:
provide stimuli to the subject via the subject interface devices;
receive inputs from the subject, via the subject interface devices, in response to the stimuli and generate interface board data indicative of the subject’s inputs simultaneously with at least one of the measurement of the dynamic vertical ground reaction forces and the center of pressure by the force plate and the capturing of the depth image data;
a depth image data collection device structured and operable to generate depth image data of the subject as the subject performs the one or more tasks simultaneously with the measurement of the dynamic vertical ground reaction forces and the center of pressure by the force plate and the receipt of subject inputs to the interface board; and
a computer-based data collection and analysis system communicatively connected to the force plate, the depth image data collection device and the interface board, and structured and operable to execute one or more point-of-care assessment system (PASS) program to provide the stimuli and collect and analyze the simultaneously generated force plate data, depth image data, and interface board data.

2. The system of claim 1 further comprising a 2D camera structured and operable to generate 2D image data of the subject as the subject performs the one or more tasks simultaneously with at least one of the measurement of the dynamic vertical ground reaction forces and the center of pressure by the force plate and the capturing of the depth image data.

3. The system of claim 1, wherein the data collection and analysis system is further structured and operable to execute the one or more PASS program comprising a skeletal tracking algorithm to measure body motion of the subject in the form of joint centers and segment orientations simultaneously with at least one of the measurement of the dynamic vertical ground reaction forces and the center of pressure by the force plate and the capturing of the depth image data.

4. The system of claim 3, wherein the data collection and analysis system is further structured and operable to execute the one or more PASS program to induce cognitive dual task activities for the subject to preform via interaction with interface board.

5. The system of claim 4, wherein the data collection and analysis system is further structured and operable to execute the one or more PASS program to induce the cognitive dual task activities for the subject to preform via interaction with interface board and force plate.

6. The system of claim 5, wherein the subject interface devices comprise a plurality of subject stimulus device and a plurality of subject input devices.

7. The system of claim 6, wherein the plurality of subject stimulus devices comprise at least one of:
a plurality of lights; and
a speaker.

8. The system of claim 7, wherein the plurality of subject input devices comprise at least one of:
at least one push button;
at least one touch screen display button;
at least one switch; and
a microphone.

9. The system of claim 6, wherein the cognitive dual task activities comprise at least one of memory games and mental tasks including mathematical computations.

10. The system of claim 6, wherein execution of the PASS program comprises implementation of at least one of a Romberg balance test, a lateral step-down task, a drop vertical jump task, a gait task, a step-down task, a sit-to-stand task, and an upper extremity pointing task.

11. A method for the assessment of neurocognitive and neuromotor performance utilizing a point-of-care assessment system (PASS), said method comprising:
generating force plate data indicative of measurement of dynamic vertical ground reaction forces on a subject’s body and of the center of pressure of the subject on the force plate as the subject performs one or more tasks on a force plate of the PASS;
providing stimuli to the subject via a plurality of subject interface devices on an interactive interface board of the PASS;
receiving inputs from the subject, via the subject interface devices, in response to the stimuli and generating interface board data indicative of the subject’s inputs simultaneously with at least one of the measurement of the dynamic vertical ground reaction forces and the center of pressure by the force plate and the capturing of the depth image data
generating depth image data of the subject, via a depth image data collection device of the PASS, as the subject performs the one or more tasks simultaneously with the measurement of the dynamic vertical ground reaction forces and the center of pressure by the force plate and the receipt of subject inputs to the interface board; and
executing one or more point-of-care assessment system (PASS) program, via a computer-based data collection and analysis system of the PASS, to provide the stimuli and collect and analyze the simultaneously generated force plate data, depth image data, and interface board data, wherein the computer-based data collection and analysis system is communicatively connected to the force plate, the depth image data collection device and the interface board.

12. The method of claim 11 further comprising generating 2D image data, via a 2D camera of the PASS, of the subject as the subject performs the one or more tasks simultaneously with at least one of the measurement of the dynamic vertical ground reaction forces and the center of pressure by the force plate and the capturing of the depth image data.

13. The method of claim 11 further comprising executing, via the data collection and analysis system, the one or more PASS program comprising a skeletal tracking algorithm to measure body motion of the subject in the form of joint centers and segment orientations simultaneously with at least one of the measurement of the dynamic vertical ground reaction forces and the center of pressure by the force plate and the capturing of the depth image data.

14. The method of claim 13 further comprising executing, via the data collection and analysis system, the one or more PASS program to induce cognitive dual task activities for the subject to preform via interaction with interface board.

15. The method of claim 14 further comprising executing, via the data collection and analysis system, the one or more PASS program to induce the cognitive dual task activities for the subject to preform via interaction with interface board and force plate.

16. A point-of-care assessment system for the assessment of neurocognitive and neuromotor performance, said system comprising:
a force plate structured and operable to generate force plate data indicative of measurement of dynamic vertical ground reaction forces on a subject’s body and of the center of pressure of the subject on the force plate as the subject performs one or more tasks;
an interactive interface board comprising a plurality of subject interface devices, wherein the interface board is structured and operable to:
provide stimuli to the subject via the subject interface devices;
receive inputs from the subject, via the subject interface devices, in response to the stimuli and generate interface board data indicative of the subject’s inputs simultaneously with at least one of the measurement of the dynamic vertical ground reaction forces and the center of pressure by the force plate and the capturing of the depth image data;
a depth image data collection device structured and operable to generate depth image data of the subject as the subject performs the one or more tasks simultaneously with the measurement of the dynamic vertical ground reaction forces and the center of pressure by the force plate and the receipt of subject inputs to the interface board;
a 2D camera structured and operable to generate 2D image data of the subject as the subject performs the one or more tasks simultaneously with the measurement of the dynamic vertical ground reaction forces and the center of pressure by the force plate and the receipt of subject inputs to the interface board; and
a computer-based data collection and analysis system communicatively connected to the force plate, the depth image data collection device, the 2D camera and the interface board, and structured and operable to execute one or more point-of-care assessment system (PASS) program to provide the stimuli and collect and analyze the simultaneously generated force plate data, depth image data, 2D data and interface board data.

17. The system of claim 16, wherein the data collection and analysis system is further structured and operable to execute the one or more PASS program comprising a skeletal tracking algorithm to measure body motion of the subject in the form of joint centers and segment orientations simultaneously with at least one of the measurement of the dynamic vertical ground reaction forces and the center of pressure by the force plate and the capturing of the depth image data.

18. The system of claim 17, wherein the data collection and analysis system is further structured and operable to execute the one or more PASS program to induce cognitive dual task activities for the subject to preform via interaction with interface board.

19. The system of claim 18, wherein the data collection and analysis system is further structured and operable to execute the one or more PASS program to induce the cognitive dual task activities for the subject to preform via interaction with interface board and force plate.

20. The system of claim 19, wherein the cognitive dual task activities comprise at least one of memory games and mental tasks including mathematical computations.
